(19) United States
(12) Plant Patent Application Publication
(10) Pub. No.: US 2013/0326768 P1
(43) Pub. Date: Dec. 5, 2013

(54) Pineapple plant named Rosé (EF2-114)

(50) ;

(71) Applicants: Ebrahim Firoozabady, Coral Gables, FL (US); Thomas R. Young, Coral Gables, FL (US)

(72) Inventors: Ebrahim Firoozabady, Coral Gables, FL (US); Thomas R. Young, Coral Gables, FL (US)

(73) Assignee: Del Monte Fresh Produce Company, Coral Gables, FL (US)

(21) Appl. No.: 13/507,101

(22) Filed: Jun. 4, 2012

(51) Int. Cl. A01H 5/00

(52) U.S. Cl. PLT/156

(57) ABSTRACT

A new pineapple variety named ‘Rosé’ is provided. Internal red or pink color with unique shell morphology and possibility of flowering control trait are traits of the new variety.

### SPECIES NAME

Ananas comosus.

### VARIETY DENOMINATION

‘Rosé’, with breeder name EF2-114.

### BACKGROUND OF THE INVENTION

A new variety of pineapple (Ananas comosus), family Bromeliaceae, has been developed using genetic engineering techniques and named ‘Rosé’. This process took 6 years; started in August 2005, using crown materials from variety MD2 (also known as Del Monte Gold pineapple), imported from Hawaii, to produce in vitro shoot cultures, introduce genes and DNA elements into leaf base sections, regenerate complete plants, perform field trials and select plants with internal pink-or red-colored fruits. The selected plants were asexually propagated in the field and via meristem culture to confirm the colored traits and other traits related to fruit and agronomic performance.

The plant is very similar to parental line, MD2, for plant and fruit characteristics and fruit internal quality. However, the internal flesh color in Rosé is pink or red, due to accumulation of lycopene in the edible part of fruit, the shell morphology is referred as “Tiger” and it might be tolerant to natural occurrence of flowering. This new variety is best suited for the fresh market and residual fruit may be processed as juice or frozen product.

The main objective of this invention was to produce a unique and differentiated variety of pineapple by accumulation of high levels of carotenoids, in particular lycopene that produces red internal color while retaining most of the characteristics of the parental line, MD2.

The invention relates to carotenoid biosynthesis in pineapple plants. More specifically, this novel pineapple was produced by genetically transforming MD2 cells in tissue culture and regenerating complete plants (Scheme 1) with expression regulators that modulate lycopene biosynthesis in the internal section of the fruit. In addition plants were transformed with genes involved in ethylene biosynthesis pathway to control flowering in the plants.

Carotenoids are isoprenoid molecules that are widespread in nature and can occur as pigments in fruits, flowers, birds, and crustaceans. Animals are unable to synthesize carotenoids de novo, and rely upon the diet as a source of these compounds. Carotenoids may contribute fundamentally to human health and in recent years there has been considerable interest in dietary carotenoids with respect to their potential in alleviating age-related diseases in humans. This attention has been mirrored by significant advances in cloning most of the carotenoid genes and in the genetic manipulation of crop plants with the intention of increasing levels in the diet.

In plants, carotenoids are essential components of the photosynthetic apparatus and are responsible for the red, orange, and yellow color of many flowers and fruit. Our understanding of carotenoid biosynthesis has advanced dramatically in recent years (Hirschberg, 2001; Fraser and Bramley, 2004). The pathway involves a series of desaturations, cyclizations, hydroxylations, and epoxidations commencing with the formulation of phytoene (See FIG. 1). A subsequent series of desaturations is responsible for lycopene synthesis. After the desaturation reactions, the cyclization of lycopene is catalyzed by two enzymes, the beta-cyclase and the zeta-cyclase, leading to the formation of beta-carotene (two beta-rings) and alpha-carotene (one beta-ring and one zeta-ring) (Cunnigham et al., 1996, 1998).

The genes of interest are derived from edible plant species, pineapple (Ananas comosus) or tangerine (Citrus reticulata). Specifically, we have over-expressed a tangerine phytoene synthase gene (Psy) (Ikoma et al, 2001). The gene is under transcriptional control of the pineapple bromelain inhibitor (BRI) gene. We have also suppressed endogenous lycopene β-cyclase (b-Lyc) and/or lycopene ε-cyclase (e-Lyc) gene expression using RNA interference (RNAi) technology in order to increase accumulation of lycopene in edible tissues of pineapple fruit (Young, T. and Firoozabady, E. 2010, U.S. Pat. No. 7,663,021). We constructed sense- and antisense-oriented sequences of the b-Lyc and e-Lyc genes derived from pineapple, which are separated by an intron of the light-inducible tissue-specific LS1 gene derived from potato (Solanum tuberosum) to form a hairpin structure. These genes are under transcriptional control of the Bromelain inhibitor gene promoter, which drives strong fruit-enhanced expression of the RNAi construct (Wintz, H-C and Firoozabady, E. 2005).

Flower initiation in pineapple can occur naturally primarily due to cool temperatures and short days. Natural flowering of pineapple plants is a major industry problem. To achieve the controlled flowering trait, we have altered expression of genes involved in ethylene biosynthesis. Ethylene is a plant hormone that plays an important role in every phase of plant development, including seed germination, fruit ripening, leaf and flower senescence, and abscission. In plants, ethylene is synthesized from the amino acid, Methionine. The immediate precursor of ethylene in higher plants is 1-aminocyclopropane-1 carboxylic acid (ACC) (Adams and Yang, 1979).

Ethylene is known to inhibit flowering in most plants. In mango and pineapple, ethylene promotes flowering (Salisbury and Ross, 1992). Ethylene, ethylene producing compounds and auxins have been used to induce flowering in commercial pineapple production (Turnbull et al., 1993).

We isolated a meristem-specific ACC synthase (flACCS) gene from pineapple and constructed sense- and antisense oriented sequences of the ACC synthase gene, which are separated by an intron of the light-inducible tissue-specific LS1 gene derived from potato (Solanum tuberosum) to form a hairpin structure for RNAi suppression of endogenous ACC synthase. The RNAi construct is under transcriptional control of the meristem-specific ACC promoter derived from pineapple.

### TRANSFORMATION METHOD

Pineapple was transformed by Agrobacterium tumefaciens-mediated transformation of organogenic tissues using a method described by Firoozabady (U.S. Pat. No. 8,049,067). To achieve both high-carotenoid and controlled flowering phenotypes, Agrobacterium strains containing either a transformation plasmid for increased carotenoid biosynthesis or for decreased ethylene biosynthesis were co-cultivated with recipient pineapple tissues. Putative transformed tissues were selected on media containing chlorsulfuron and subsequently screened for the presence of target genes by PCR.

A. tumefaciens, [Strain GV31011] (Koncz and Schell, 1986), is a disarmed Agrobacterium strain commonly used for the delivery of T-DNA into plant cells. Different genes were inserted into T-DNA in a binary vector (see FIG. 2) for introduction to pineapple. The Psy derived from Citrus reticulata, tangerine (Ikomaa et al, 2001), encodes an enzyme that coverts geranylgeranyl pyrophosphate (GGPP) to cis-phytoene, an intermediate in lycopene and beta-carotene biosynthesis.

The lycopene beta-cyclase gene (b-Lyc) derived from Ananas comosus, pineapple, encodes an enzyme that coverts lycopene to gamma-carotene, a metabolic precursor of beta-carotene.

The lycopene epsilon-cyclase gene (e-Lyc) derived from Ananas comosus, pineapple, encodes an enzyme that coverts lycopene to sigma-carotene, a metabolic precursor of alpha-carotene.

The modified acetolactate synthase (Chaleff, R. S., and Mauvais, C. J., 1984) (ALS) gene (surBHRA) derived from Nicotiana tabacum, tobacco, catalyzes the biosynthesis of branched chain amino acids even in the presence of chlorsulfuron (Lee, K. et al., 1988), which allows for the selection of transformed pineapple cells.

Plasmid pHCW1 used for pineapple transformations was constructed by the laboratory of Del Monte Fresh Produce Company, Richmond, Calif. pHCW1 contains a tetracycline resistance gene (tetRA) from plasmid RP1 and the origin of replication from plasmid pACYC, which allows for selection and maintenance in Escherichia coli and the pVS1 replicon derived from Pseudomonas aeruginosa, which ensures replication in Agrobacterium tumefaciens. pHCW1 contains the 25-base pair sequences that delimit the T-DNA transfer and a 110-base pair synthetic sequence between the borders that forms multiple cloning restriction sites to allow integration of different T-DNA cassettes (see Table 1).

The plasmid pCHW1 was used to create pHCW.T-7 and pHCWflACC3′-2 binary vector plasmids. Binary vectors were transferred to disarmed A. tumefaciens strain GV3101. The GV3101 with pHCW.T-7 vector was named AG76 and the one with pHCWflACC3′-2 was named AG62 (see FIG. 3). AG76 and AG62 were mixed together and used for pineapple transformation. Genetic elements of the vectors are described in Tables 2 and 3.

Genetic engineering of the MD2 took place in the Laboratory of Del Monte Fresh Produce Company in Richmond, Calif., USA, where transgenic plants were produced and propagated in tissue culture. Then the propaguls were taken to the research area of Corporacion de Desarrollo Agricola Del Monte, S.A. (Pindeco), Buenos Aires-Puntarenas, Costa Rica, for field evaluation, propagation in the field and in the laboratory for mass propagation of the variety.

### SUMMARY OF THE INVENTION

A new variety of pineapple (Ananas comosus), family Bromeliaceae, has been developed using genetic engineering techniques and named ‘Rosé’ or EF2-114. Using crown materials from variety MD2 (also known as Del Monte Gold pineapple) to produce in vitro shoot cultures, introduce genes and DNA elements into leaf base sections, regenerate complete plants, perform field trials and select plants with internal pink- or red-colored fruits. The selected plants were asexually propagated in the field and via meristem culture to confirm the colored traits and other traits related to fruit and agronomic performance. The invention relates to production of a new and distinct variety of the Bromeliaceae, or pineapple family.

The new plant variety EF2-114 is characterized by pink or red flesh color and “Tiger” shell color, when compared with the parental line, MD2, and it might be tolerant to natural occurrence of flowering.

Internal color of fruits can be variable depending on the stage of ripening (FIG. 4). The tiger trait was defined as the color in shell has in the shoulder of each fruitlet a combination of colors green, yellow, orange (and red) due to expression of carotenoid genes in the shell (FIGS. 5-7). The plant morphology generally is the same as MD2, the parental plant (FIGS. 8-11).

### BOTANICAL DESCRIPTION OF THE PLANT

The description of the new variety is based on observations of well fertilized specimens which were grown under field conditions, in the Buenos Aires region, Costa Rica, where temperatures generally range from 14° C. to 37° C., and annual rainfall averages 3251 mm.

The plants were grown at a research facility in Buenos Aires-Puntarenas, Costa Rica.

All fruit and plant characteristics including color terminology and color designations reported herein are in accordance with Brazilian Descriptors for MD2 (Table 4). Essentially, “Rosé” is same as MD2 for all fruit and plant characteristics with the exception of fruit internal color, Tiger trait and possibly flowering control trait. See attached Brazilian Descriptors for MD2.
Plant identification:
   Name.—Ananas comosus.
   Parentage.—MD2.
   Origin.—Genetic engineering of MD2 followed by selection in the field trials for the traits of interest.
   Botanic.—Bromeliaceae or pineapple family. Subfamily: Bromelioideae. Genus: Ananas. Subgenus: comosus. Variety: “Rosé”, breeder's name EF2-114.
   Commercial.—Bromeliad fruit plant.
General:
   Fertility.—As any other grown up pineapple, this plant is self-incompatible. For this reason, seeds are rarely observed. Commercial propagation is via vegetative propagules (suckers, stem shoots and slips) and fruit crowns.
   Vigor.—It is considered that the plant vigor is similar as to mother plants, MD2.
   Yield.—Estimated yield is same as MD2, 120-125 MT/ha in Plant Crop and 95-100 MT/ha in Ratoon Crop.
   Market.—Fruit will be designated to the international fruit market and commercialized into the fresh fruit market. Residual fruit may be processed as juice or frozen product.

### REFERENCES

1. Adams D. O., Yang S. F. 1979. Ethylene biosynthesis: Identification of 1-aminocyclopropane-1-carboxylic acid as an intermediate in the conversion of methionine to ethylene. Proc Natl Acad Sci U S A.; 76(1):170-174.

2. Barker, R., Idler, K., Thompson, D., Kemp, J. (1983) Nucleotide sequence of the T-DNA region from the Agrobacterium tumefaciens octopine Ti plasmid pTi15955. Plant Mol. Biol. 2:335-350.

3. Chaleff, R. S., and Mauvais, C. J. (1984) Acetolactate synthase is the site of action of two sulfonylurea herbicides in higher plants. Science 224:1443-1445.

4. Chang, A. C. Y. & Cohen, S. N. (1978). Construction and characterisation of amplifiable multicopy DNA cloning vehicles derived from the p15a cryptic miniplasmid. J Bacteriol 134, 1141-1156

5. Cunningham, F. X., Pogson, B. J., Sun, Z., McDonald, K., DellaPenna, D., and Gantt, E. (1996). Functional analysis of the beta and epsilon lycopene cyclase enzymes of Arabidopsis reveals a mechanism for control of cyclic carotenoid formation. Plant Cell 8, 1613-1626.

6. Cunningham, F. X., Jr., and Gantt, E. (1998). Genes and enzymes of carotenoid biosynthesis in plants. Arum. Rev. Plant Physiol. Plant Mol. Biol. 49,557-583.

7. DePicker, A., Stachel, S., Dhaese, P., Zambryski, P., Goodman, H. M. (1983) Nucleotide sequences and transcript map of the Agrobacterium tumefaciens Ti plasmid-encoded octopine synthase gene. J. Mol. Appl. Genet. 1:499-511.

8. Eckes, P., Rosahl, S., Schell, J. and Willmitzer, L., (1986) Isolation and characterization of a light-inducible, organ-specific gene from potato and analysis of its expression after tagging and transfer into tobacco and potato shoots, Mol. Gen. Genet. 205,14-22

9. Firoozabady, E. U.S. Pat. No. 8,049,067, Organogenic transformation and regeneration.

10. Fraser, P. D., & Bramley, P. M. (2004). The biosynthesis and nutritional uses of carotenoids. Progress in lipid research, 43(3), 228-65

11. Hirschberg, J. (2001). Carotenoid biosynthesis in flowering plants. Curr. Opin. Plant Biol. 4,210-218.

12. Hirschberg, I, M. Cohen, M. Harker, T. Lota, V. Mann, and I. Pecker. 1997. Molecular genetics of carotenoid biosynthesis pathway in plants and algae. Pure Appl. Chem. 69:2145-2150.

13.

14. Ikomaa, Yoshinori, Akira Komatsub, Masayuki Kitac, Kazunori Ogawad, Mitsuo Omurac, Masamichi Yanoc and Takaya Moriguchi, (2001) Expression of a phytoene synthase gene and characteristic carotenoid accumulation during citrus fruit development. Physiologia Plantarum 111: 232-238.

15. Itoh, Y., Watson, J. M., Haas, D., Leisinger, T. (1984) Genetic and molecular characterization of the Pseudomonas plasmid pVS1. Plasmid 11:206-220.

16. Koncz C, and Schell J. (1986) The promoter of TL-DNA gene 5 controls the tissue-specific expression of chimaeric genes carried by a novel type of Agrobacterium binary vector. Mol Gen Genet 204: 383-396

17. Lee, K. et al., (1988) The molecular basis for sulfonylurea herbicide resistance in tobacco. EMBO J. 7:1241-1248.

18. Neuteboom L. W., Kunimitsu W. Y., Webb D, Christopher D. A., 2002. Characterization and tissue-regulated expression of genes involved in pineapple (Ananas comosus L.) root development. Plant Science, 165:1021-1035.

19. Salisbury, F. B. and Ross, C. W. 1992. Plant Physiology, 4th ed., Wadsworth.

20. TURNBULL, C. G. N.; NISSEN, R. J.; SINCLAIR, E. R.; ANDERSON, K. L.; SHORTER, A. J. Ethephon and causes of flowering failure in pineapple. Acta Horticulturae, Honolulu, n.334, p. 83-88, out, 1993.

21. Young, T. and Firoozabady, E. 2010, U.S. Pat. No. 7,663,021. Transgenic pineapple plants with modified carotenoid levels and methods of their production

22. Waters, S. H., Rogowski, P., Ginsted, J., Altenbuchner, J., and Schmidt, R. (1983) The tetracycline resistance determinants of RP1 and Tnl 721; nucleotide sequence analysis. Nuc. Acids Res. 11(17):6089-6105.

23. Wintz, H-C and Firoozabady, E. 2005, patent PUBLICATION NUMBER-2006113709/WO-A2 Plant promoters, terminators, genes, vectors and related transformed plants.

### FIGURE LEGENDS

FIG. 1. Enzymes and genes in the carotenoid biosynthesis pathway in plants and algae. (Adapted from J. Hirschberg et al., Pure & Appl. Chem. 69(10):2151-58). Genes used in pineapple transformations to create EF2-114 are shown in the boxes. Psy is phytoene synthase gene from tangerine and b-lyc and e-Lyc are partial sequences of pineapple lycopene β-cyclase and lycopene ε-cyclase genes respectively.

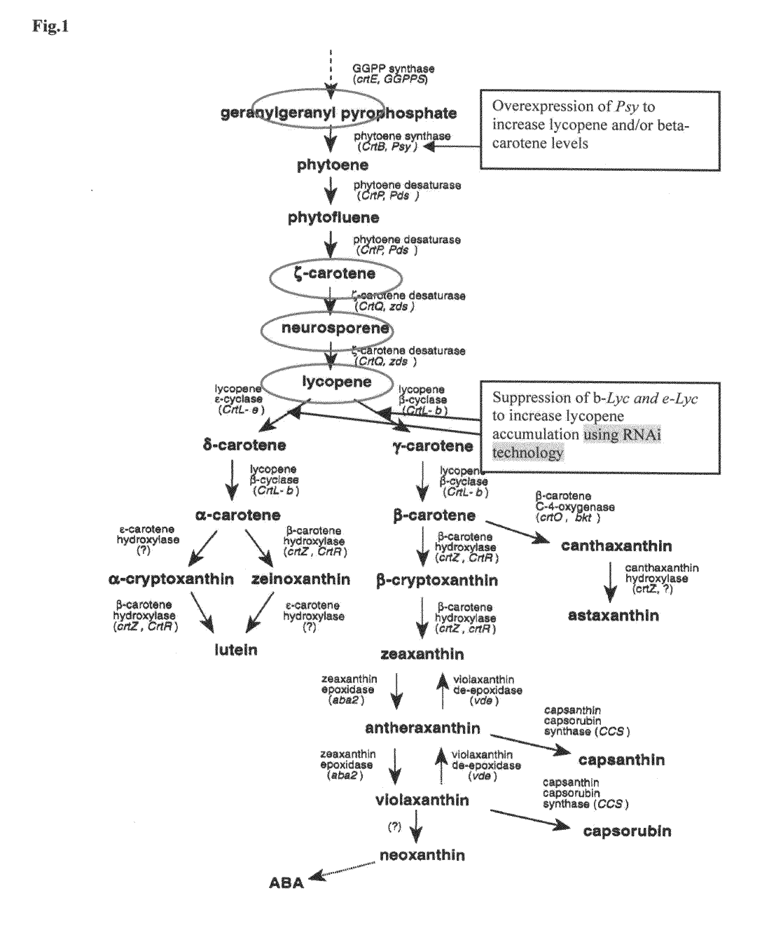

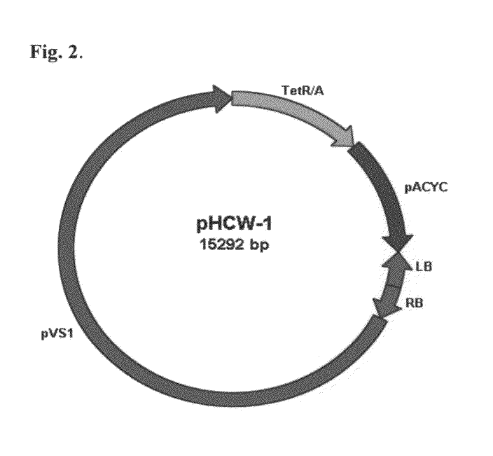

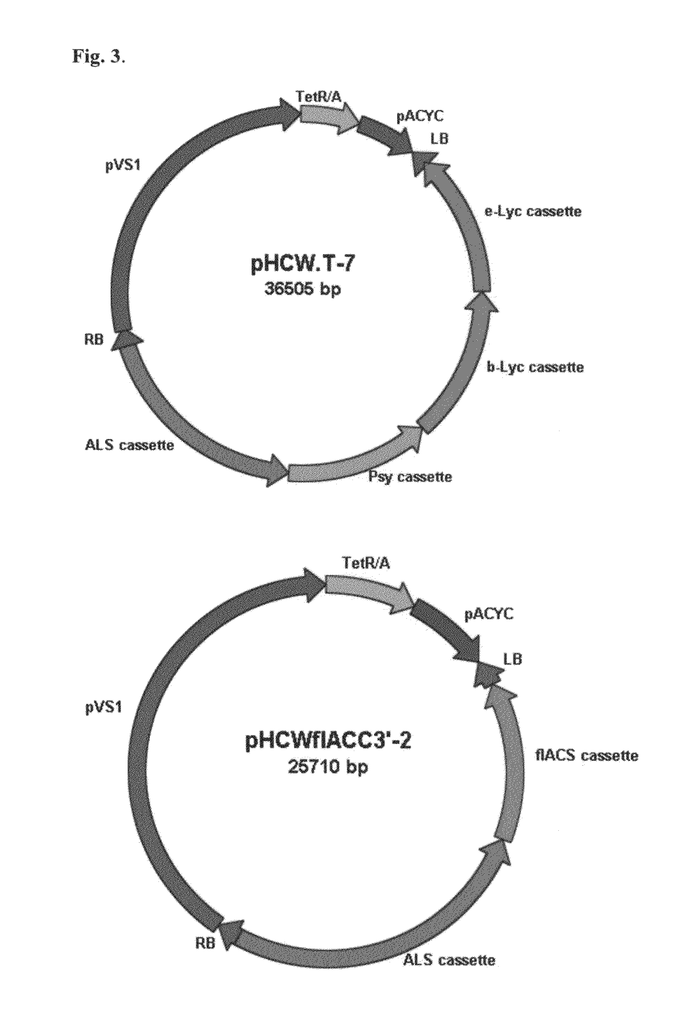

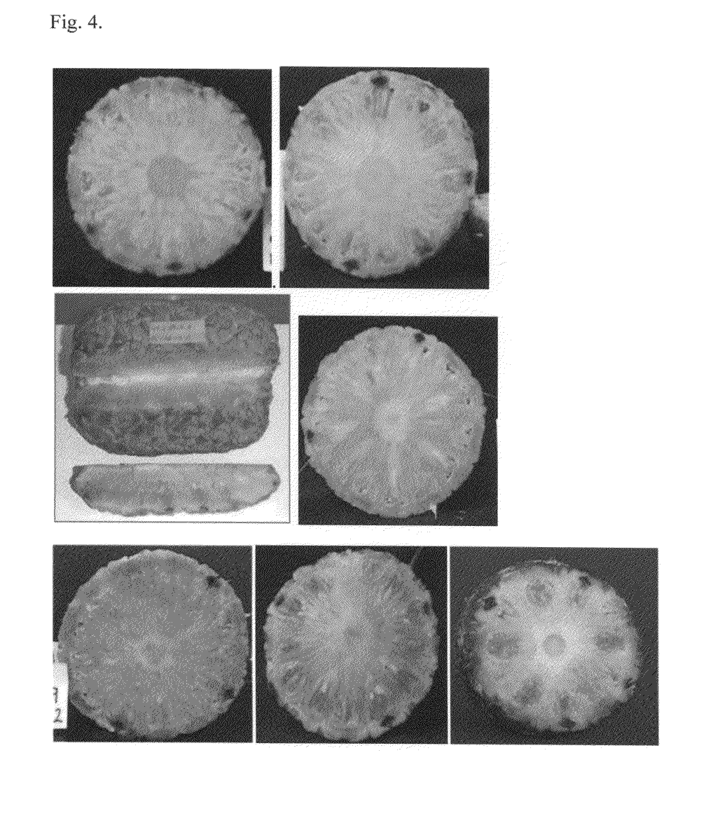

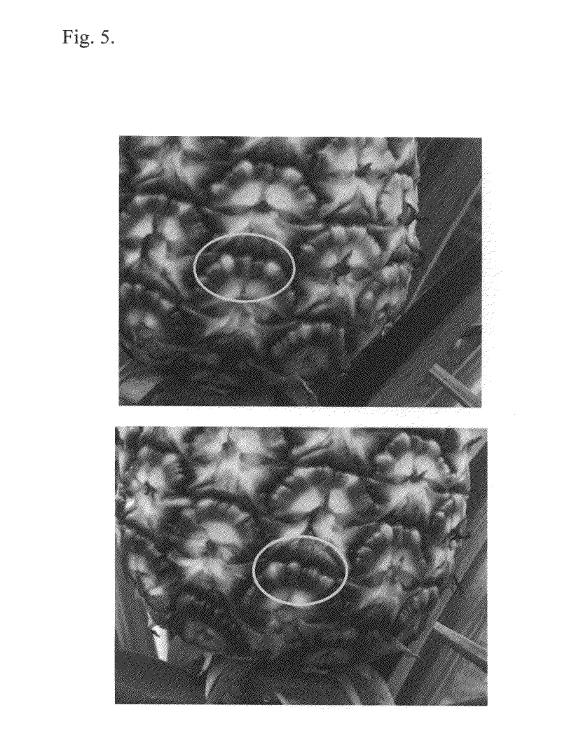

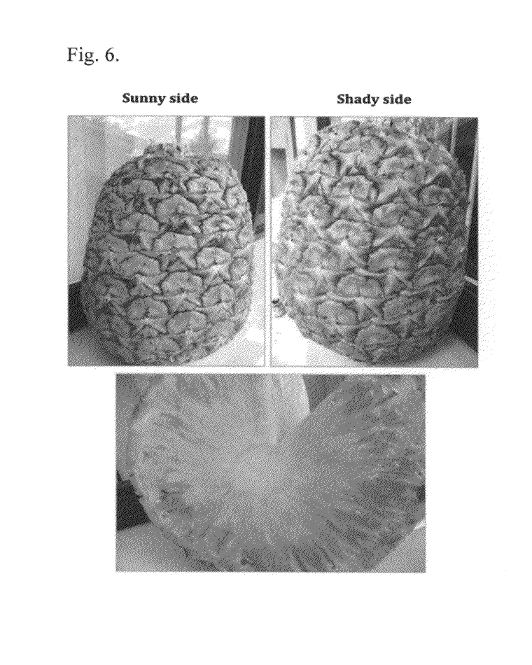

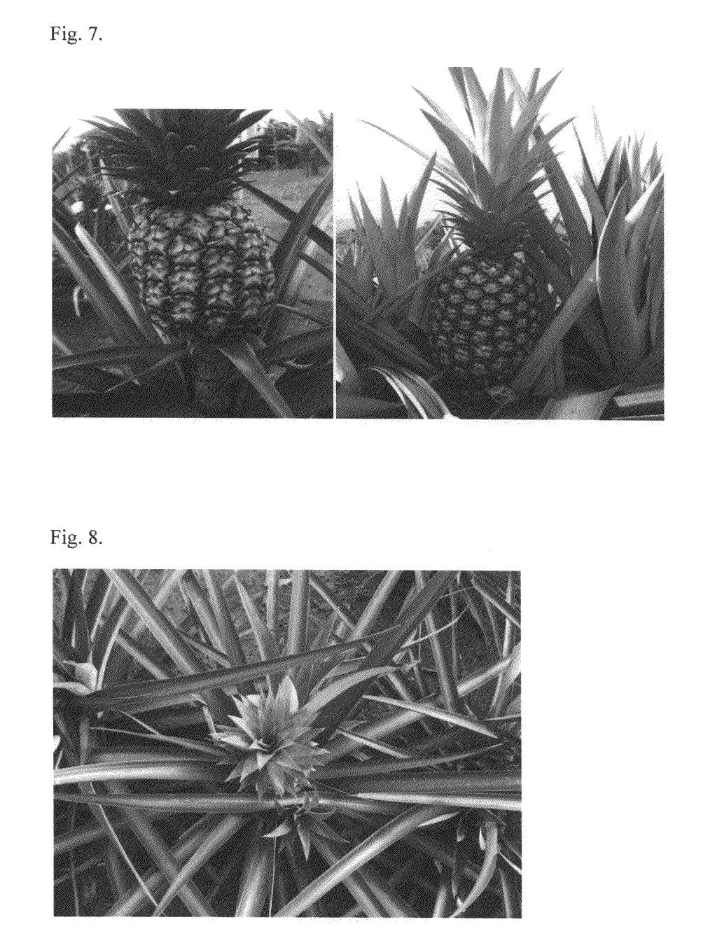

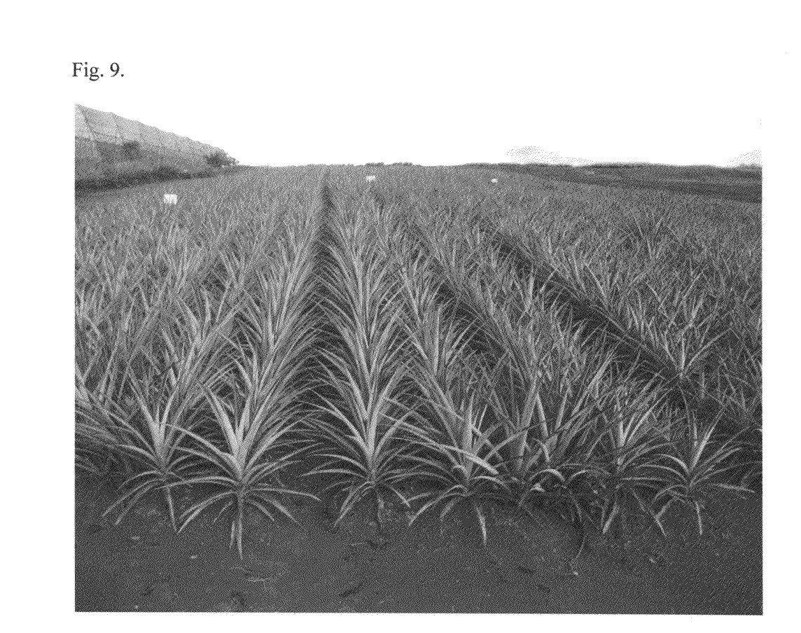

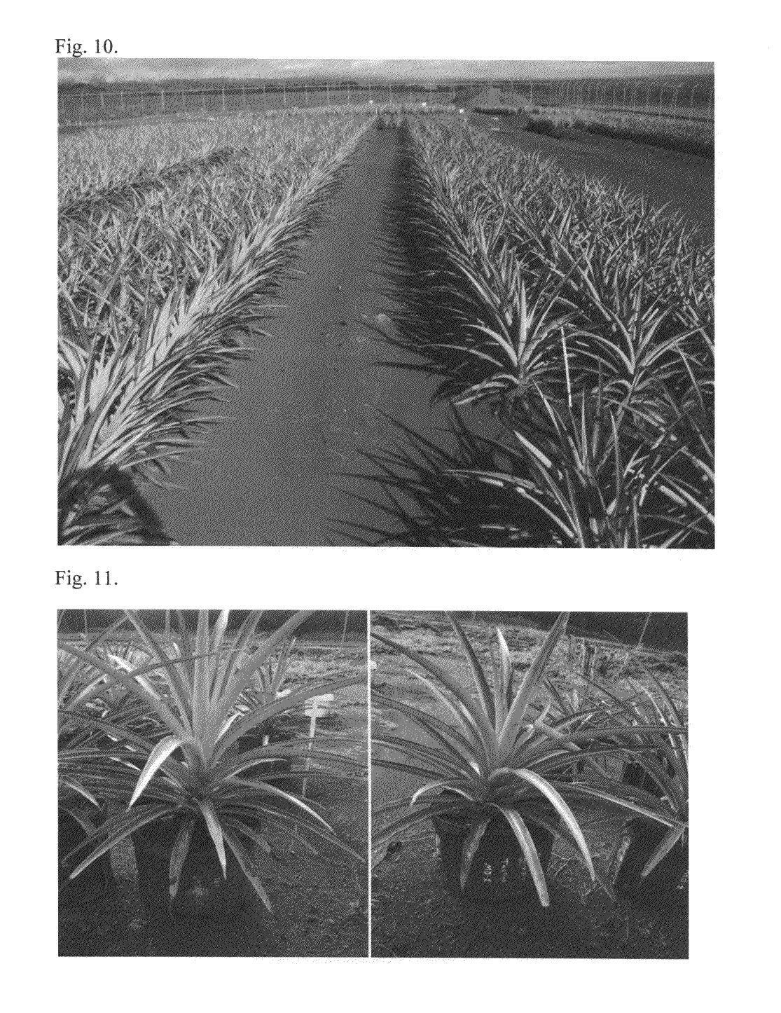

FIG. 2. Map of plasmid binary vector backbone, pHCW1, used for genetic engineering of pineapple. For definition of genetic elements see Table 1.
RB—Right Border
pVS 1—Agrobacterium origin of replication
TetR/A—Tetracyclin gene (bacterial selectable marker)
pACYC—Bacterial origin of replication
LB—Left Border

FIG. 3. Plasmid pHCW1 was used to create pHCW.T-7 binary vector (in AG76) and pHCWflACC3′-2 binary vector (in AG62). For definition of genetic elements see Table 2 and 3.
Vector Backbone:
RB: Right T-DNA Border
pVS1: Agrobacterium origin of replication
TetR/A: Tetracyclin gene (bacterial selectable marker)
pACYC: Bacterial origin of replication
LB: Left T-DNA Border
T-DNA
ALS cassette: EHS-Ubp (promoter)—ALS-ALS3′ terminator
Psy casssette: BRIP (promoter)-Psy-Ubp terminator
bLyc cassette: BRIP (promoter)-bLyc (+)-LS 1 intron-bLyc(−)-Ubp terminator
eLyc cassette: BRIP (promoter)-eLyc(+)-LS 1 intron-eLyc(−)-Ubp terminator
flACS cassette: Ubp(promoter)-flACS(+)-LS1 intron-flACS(−)-Ubp terminator

FIG. 4. Shows the internal color of the fruits being pink to red due to accumulation of lycopene.

FIG. 5. Shows “Tiger” shell color in EF2-114 fruits. The color in shell has in the shoulder of each fruitlet a combination of colors green, yellow, orange (and red).

FIG. 6. A “Tiger” fruit showing shell and internal color at harvest.

FIG. 7. Shows an immature fruit of ‘Rosé’ with Tiger shell morphology (left) and an immature fruit of MD2 (right) both ˜145 days after forcing.

FIG. 8. Shows overhead view of ‘Rosé’ EF2-114 plant. FIG. 9. Shows a ‘Rosé’ EF2-114 plantation. FIG. 10. Shows EF2-114 (left) and MD2 (right) experimental plots. FIG. 11. Shows EF2-114 (left) and MD2 (right) meristem culture-derived plants. After 15 weeks growth in greenhouse, ˜15 cm long plants were transplanted in pots, and the pictures were taken 11 months later.

TABLE 1
Genetic Elements of plasmid pHCW1

| Genetic | |
|---|---|
| Element | Source and Function |
| RB | A 25 bp nucleotide sequence that acts as the initial point of DNA |
| | transfer into plant cells which was originally isolated from |
| | pTiT37 (Depicker et al., 1982). |
| pvs1 | The pVS1 replicon derived from Pseudomonas aeruginosa, |
| | which ensures replication in Agrobacterium tumefaciens |
| | (Itoh et al., 1984). |
| TetRA | A tetracycline resistance gene from plasmid RP1, which allows |
| | for selection of the binary plasmid in Agrobacterium tumefaciens |
| | and Escherichia coli (Waters et al., 1983). |
| pACYC | The origin of replication from plasmid pACYC, which ensures |
| | replication in Escherichia coli (Chang et al., 1979). |
| LB | A 25-nucleotide sequence that delimits the T-DNA transfer and |
| | acts as the endpoint of DNA transfer into plant cells. It was |
| | originally isolated from TiA6 (Barker et al., 1983). |
| MCS | A 110-nucleotide sequence that synthetically was created, which |
| | is composing of multiple cloning restriction sites in order to allow |
| | integration of different cassettes into the plasmid. |

TABLE 2
Genetic Elements of T-DNA in plasmid pHCW.T-7 in AG76

| Genetic | Size | |
|---|---|---|
| Element | (Kb) | Function (Reference) |
| BRIp2.5 | 2.5 | A promoter derived from the bromelain inhibitor |
| | | (BRI) gene from Ananas comosus that drives fruit- |
| | | enhanced expressionof the target gene(s). |
| Psy | 1.131 | A phytoene synthase (Psy) gene from Citrus |
| | | reticulata, identical to the gene isolated from |
| | | mandarin fruit (Ikoma et al. 2001), which encodes |
| | | an enzyme in carotenoid biosynthesis. |
| Ubpter | 0.94 | A terminator derived from the polyadenylation |
| | | sequence of the ubiquitin (Ubi) gene from Ananas |
| | | comosus terminates transcription of the transgene(s). |
| BRIp2.5 | 2.5 | A promoter derived from the promoter sequence |
| | | from the bromelain inhibitor (BRI) gene from |
| | | Ananas comosus that drives fruit-enhanced |
| | | expression of the RNAi transgene. |
| eLyc sense | 0.504 | A fragment of the lycopene ε-cyclase gene from |
| fragment | | Ananas comosus in sense orientation, which is used |
| | | in an RNAi expression system to down-regulate |
| | | endogenous lycopene ε-cyclase, an enzyme in |
| | | carotenoid biosynthesis. |
| ST-LS1 | 0.193 | An intron of the light-inducible tissue-specific |
| | | ST-LS1 gene from Solanum tuberosum that |
| | | functions as a spacer between sense and antisense |
| | | gene fragments enhancing vector stability. |
| eLyc | 0.504 | A fragment of the lycopene ε-cyclase gene from |
| antisense | | Ananas comosus in antisense orientation, which is |
| fragment | | used in an RNAi expression system to down- |
| | | regulate endogenous lycopene ε-cyclase, an |
| | | enzyme in carotenoid biosynthesis. |
| Ubpter | 0.94 | A terminator derived from the polyadenylation |
| | | sequence of the ubiquitin (Ubi) gene from Ananas |
| | | comosus terminates transcription of the RNAi |
| | | transgene. |
| BRIp2.5 | 2.5 | A promoter derived from the promoter sequence |
| | | from a bromelain inhibitor (BRI) gene from Ananas |
| | | comosus that drives fruit-enhanced expression of |
| | | the RNAi transgene. |
| b-Lyc | 0.619 | A fragment of the lycopene β-cyclase gene from |
| sense | | Ananas comosus in sense orientation, which is used |
| fragment | | in an RNAi expression system to down-regulated |
| | | endogenous lycopene β-cyclase, an enzyme in |
| | | carotenoid biosynthesis. |
| ST-LS1 | 0.193 | An intron of the light-inducible tissue-specific |
| | | ST-LS1 gene from Solanum tuberosum (Eckes |
| | | et al, 1986) that functions as a spacer between |
| | | sense and antisense gene fragments enhancing |
| | | vector stability. |
| b-Lyc | 0.619 | A fragment of the lycopene β-cyclase gene from |
| antisense | | Ananas comosus in antisense orientation, which |
| fragment | | is used in an RNAi expression system to down- |
| | | regulated endogenous lycopene β-cyclase, an |
| | | enzyme in carotenoid biosynthesis. |
| Ubpter | 0.94 | A terminator derived from the polyadenylation |
| | | sequence of the ubiquitin (Ubip) gene from |
| | | Ananas comosus terminates transcription of the |
| | | RNAi transgene. |

Selectable Marker

| | | |
|---|---|---|
| EHS1.7- | 4.257 | A promoter derived from the epoxide hydrolase |
| Ubp1.5 | | (EHS) gene fused to the ubiquitin (Ubip) gene |
| | | promoter and the native intron from Ananas |
| | | comosus that drives constitutive expression of |
| | | the selectable marker gene. |
| EHS1.7 | 1.7 | A promoter derived from the epoxide hydrolase |
| | | (EHS) gene from Ananas comosus (Neuteboom |
| | | et al, 2002) that drives constitutive expression |
| | | of the selectable marker gene. |
| Ubp1.5 | 1.5 + | A 1.5 kb tetrameric ubiquitin gene promoter from |
| | 1.057 | pineapple (Ananas comosus), which drives |
| | | constitutive expression of the selectable marker |
| | | gene. The promoter includes an endogenous 1057 |
| | | bp intron sequence. |
| ALS | 1.17 | A mutant acetolactate synthase gene from tobacco |
| | | (Nicotiana tabacum), which confers resistance to |
| | | chlorsulfuron and allows selection of transformed |
| | | plant cells (Chaleff and Mauvais, 1984; Lee et al., |
| | | 1988). |
| ALS 3′ | 2.7 | An endogenous terminator derived from untrans- |
| | | lated polyadenylation signal of the acetolactate |
| | | synthase (ALS) gene from Nicotiana tabacum |
| | | (Chaleff and Mauvais, 1984; Lee et al., 1988). |

TABLE 3
Genetic Elements of T-DNA in plasmid pHCWflACC3′-2 in
AG62. Elements are the same as above (Table 2) except those
mentioned below.

| Genetic | Size | |
|---|---|---|
| Element | (Kb) | Function (Reference) |
| flACS(+)sense | 0.406 | A 406 bp 3′sequence of meristem-specific ACC |
| | | synthase gene, isolated from pineapple (A. |
| | | comosus, in the sense orientation, which is |
| | | used in an RNAi expression system to down- |
| | | regulate endogenous ACC synthase, a key |
| | | enzyme in ethylene biosynthesis, thereby |
| | | producing a plant that displays improved |
| | | characteristics such as delayed flowering. |
| flACS(−) | 0.406 | A 406 bp 3′sequence of meristem-specific ACC |
| antisense | | synthase gene, isolated from pineapple (A. |
| | | comosus), in the antisense orientation. |

Scheme 1: production of EF2-114 pineapples.

| Stage | Date (time) |
|---|---|
| Immature MD2 fruits (40 days prior to harvest) | Aug. 10, 2005 |
| arrived from Hawaii | |
| Meristems isolated from the crown and cultured | Aug. 17, 2005 |
| Established shoot culture and propagate in vitro | Aug. 17, 2005 to Sep. |
| | 20, 2006 |
| Longitudinal sections of shoots were prepared and | Sep. 20, 2006 to Sep. |
| cultured | 29, 2009 |
| Leaf base sections were prepared and mixed with | Sep. 29, 2006 to Oct. |
| Agrobacterium strains AG-62 and AG-76 and | 02, 2006 |
| cocultivated for three days | |
| Leaf bases were transferred to recovery media | Oct. 2, 2006 to Oct. |
| containing carbenicillin (to kill off | 27, 2006 |
| Agrobacterium) | |
| Leaf bases were transferred to selection media | Oct. 27, 2006, Nov. |
| containing antibiotics (to kill off residual | 2, 2006, Nov. 14, 2006, |
| Agrobacterium) and chlorsulfuron, CS (an | Dec. 4, 2006, Jan. 3, |
| herbicide), to select for transformed plant cells | 2007, Jan. 29, 2007, |
| and produce transformed organogenic materials. | Feb. 15, 2007 |
| A total of 7 transfers were done with increasing | |
| selection pressure to produce pure transgenic tiny | |
| buds. | |
| Tiny bud clusters were transferred (two times) to | Mar. 13, 2007, Mar. |
| media containing CS and antibiotics to further | 29, 2007 |
| grow and multiply transformed shoots and shoot | |
| buds | |
| Shoots and shoot buds were transferred to media | Apr. 10, 2007 |
| containing high level of Agar (8 and 10 g/l) to | |
| normalize transformed shoots and shoot buds | |
| Shoot clusters were transferred to media with CS, | May 4, 2007 to Dec. |
| antibiotics, Agar (7-10) and hormones to | 14, 2007 |
| normalize and propagate shoots. | |
| An event referred to as 16.184.1 included 14 lines | Jan. 21, 2008 |
| were subcultured in liquid media for further | |
| growth | |
| 9 lines or groups of the 16.184.1 event (consisting | Feb. 14, 2008 |
| of 349 shoots) were shipped to Pindeco for field | |
| trials. In Pindeco the 9 groups were numbered as | |
| 109-117 and were grown in the greenhouse for | |
| further growth and filed trials. | |
| 2 additional lines or groups of the 16.184.1 event | Aug. 1, 2008 |
| (consisting of 85 shoots) were shipped to Pindeco | |
| for field trials. In Pindeco the 2 groups were | |
| numbered as 208 and 209 and were grown in the | |
| greenhouse for further growth and filed trials. | |
| PCR analyses confirmed that the genes are | Aug. 26, 2008 |
| present in tissue culture-derived event shoot | |
| samples | |
| 2 additional lines or groups of the 16.184.1 event | Jan. 26, 2009 |
| (consisting of 150 shoots) were shipped to | |
| Pindeco for field trials. In Pindeco the 2 groups | |
| were numbered as 345 and 346, and were grown | |
| in the greenhouse for further growth and filed | |
| trials. | |
| Plants were grown in the GH for 15-20 weeks | 2009-2012 |
| before transferred to soil in the field trials. Fruits | |
| were cut for internal color evaluation. During the | |
| field trials groups 109, 110, 114, 116, 208, and | |
| 346 exhibited pink or red internal colors. Internal | |
| quality (Brix, citric acid, ascorbic acid and pH) | |
| were measured on the colored fruits and | |
| propaguls were grown for future generations' | |
| evaluation. Molecular analyses (PCR and | |
| Southern) confirmed the presence of the genes. | |
| Southern further confirmed that all these group | |
| are one transgenic even and collectively are | |
| referred as EF2-114 | |

TABLE 4
MINIMUM DESCRIPTORS FOR PINEAPPLE (Ananas comosus
(L.) Merrill)
Proposed name for the plant variety MD-2

| | | Code for |
|---|---|---|
| | | each |
| | Description of the | description |
| Characteristic | characteristic | MD-2 |
| 1. Plant: attitude | semi-erect | |
| (+) | intermediary | |
| | open | 7 |
| 2. Plant: number of active | low | |
| leaves | medium | 5 |
| (+) | high | |
| 3. Leaf: length | short | |
| (+) | medium | 5 |
| | long | |
| 4. Leaf: width | narrow | |
| (+) | medium | 5 |
| | broad | |
| 5. Leaf predominant color on | light green | |
| upper face | dark green | 2 |
| | purple-green | |
| | green purple | |
| | purple | |
| | vred | |
| 6. Leaf: anthocyanin coloration | absent | 1 |
| | present | |
| 7. Leaf: variegation | absent | 1 |
| | present | |
| 8. Leaf: spines | absent | |
| (+) | inconspicuos | 2 |
| | conspicuos | |
| 9. Leaf: distribution of spines at | at base only | |
| margin | at apex only | 2 |
| (+) | at base and apex | |
| | regular | |
| | irregular | |
| 10. Inflorescense: number of | low | |
| flowers | medium | 5 |
| (+) | high | |
| 11. Flower: predominant | whitish | |
| coloration of sepal | greenish | |
| | purplish | 3 |
| 12. Flower: petals base | free | 1 |
| | fused | |
| 13. Flower: imbricate petals | absent | |
| | present | 2 |
| 14. Flower: predominant | whitish | |
| coloration of petal tip | light purple | |
| | medium purple | 5 |
| | dark purple | |
| 15. Flower: ratio of white color | low | 3 |
| at petal | medium | |
| (+) | high | |
| 16. Flower: disposition of anthers | separate | |
| | grouped | 2 |
| 17. Flower: number of pollen at | low | 3 |
| the anthers | medium | |
| | high | |
| 18. Flower: length of style | short | |
| (+) | medium | 2 |
| | long | |
| 19. Suckers: number | low | |
| (+) | medium | 5 |
| | high | |
| 20. Peduncle: length | short | 3 |
| (+) | medium | |
| | long | |
| 21. Peduncle: diameter at the | small | |
| middle portion | medium | 5 |
| (+) | large | |
| 22. Peduncle: number of | (absent or very low) | |
| younglets/bulbs/slips | (low) | |
| (+) | (medium) | 3 |
| | (high) | |
| | (very high) | |
| 23. Peduncle: number of bracts | low | |
| | medium | 5 |
| | high | |
| 24. Peduncle: imbricate bracts | absent | |
| | present | 2 |
| 25. Peduncle: trichomes | absent | |
| | present | 2 |
| 26. Fruit: length | short | |
| (+) | medium | 5 |
| | long | |
| 27. Fruit: diameter at base | small | |
| (+) | medium | |
| | large | 7 |
| 28. Fruit: diameter at the middle | small | |
| portion | medium | |
| | large | 7 |
| 29. Fruit: diameter of tip | small | |
| (+) | medium | |
| | large | 7 |
| 30. Fruit: shape | conic | |
| (+) | conic to cylindric | |
| | cylindric | 3 |
| | eliptic | |
| | global/spheric | |
| 31. Fruit: color before | silvery green | |
| physiologic maturity, with | light green | |
| the fruit completely shaped | medium green | |
| | dark green | 4 |
| | red | |
| | r purple | |
| 32. Fruit: color of skin at the | (green) | |
| point to consume | (green with yellow spots) | |
| | (light yellow) | |
| | (golden yellow) | |
| | (redish orange) | 4 |
| | (purple) | |
| 33. Fruit: color homogeneity of | absent | |
| the skin | present | 2 |
| 34. Fruit: number of fruit basal | (absent or very low) | 1 |
| slips | (low) | |
| (+) | (medium) | |
| | (high) | |
| | (very high) | |
| 35. Fruit: detachable fruitlets | absent | 1 |
| | present | |
| 36. Fruit: relief of fruitlet | flat | 1 |
| | prominent | |
| | very prominent | |
| 37. Fruit: color of flesh | white to cream | |
| | yellow | |
| | golden yellow | 3 |
| | orange | |
| 38. Fruit: firmness of flesh | soft | |
| | medium | |
| | firm | 7 |
| 39. Fruit: fibrousness in the flesh | low | |
| | medium | 5 |
| | high | |
| 40. Fruit: succulence | low | |
| | medium | 5 |
| | high | |
| 41. Fruit: diameter of central | small | |
| axis | medium | |
| (+) | large | 7 |
| 42. Fruit: concentration of | low | |
| soluble solids (Brix degrees) | medium | 5 |
| (+) | high | |
| 43. Fruit: acidity (fixed in | low | |
| percentage) | medium | 5 |
| (+) | high | |
| 44. Fruit: flavor | weak | |
| | medium | 5 |
| | strong | |
| 45. Crown: position | erect | |
| | open | 3 |
| | decumbent | |
| 46. Crown: length | short | |
| (+) | medium | 5 |
| | long | |
| 47. Crown: weight | low | |
| (+) | medium | |
| | high | 7 |
| 48. Crown: tendency to multiple | (absent or very low) | |
| crowns | (low) | |
| | (medium) | 3 |
| | (high) | |
| | (very high) | |

COMPLEMENTARY CHARACTERISTIC

| | | |
|---|---|---|
| 49. Resistance to fusarium | (high resistance) | |
| (Fusarium subglutinans) | (resistant) | |
| | (medium resistance) | |
| | (medium susceptibility) | |
| | (susceptible) | |
| | (high susceptibility) | 4 |

(+) See item “VI. OBSERVATIONS AND FIGURES”.

What is claimed is:

1. A new and distinct variety of Ananas comosus plant named ‘Rosé’ with breeder name EF2-114 as shown and described herein.
